(19) United States
(12) Patent Application Publication
(10) Pub. No.: US 2026/0102165 A1
(43) Pub. Date: Apr. 16, 2026

(54) SYSTEMS AND METHODS FOR DELIVERY OF MAGNETIC ANASTOMOSIS RELATED DEVICES USING SURGICAL ROBOTS

(71) Applicant: G.I. Windows, Inc., Westwood, MA (US)

(72) Inventors: Jonathan P. Boduch, Wrentham, MA (US); Michael Boutillette, Sudbury, MA (US)

(21) Appl. No.: 19/323,319

(22) Filed: Sep. 9, 2025

Related U.S. Application Data
(63) Continuation-in-part of application No. PCT/US25/23903, filed on Apr. 9, 2025.
(60) Provisional application No. 63/632,725, filed on Apr. 11, 2024.

(51) Int. Cl. A61B 17/115; A61B 17/00; A61B 34/00; A61B 34/30; A61B 34/37

(52) U.S. Cl. CPC A61B 17/115; A61B 34/37; A61B 34/73; A61B 2017/00876; A61B 2034/302

(57) ABSTRACT

Certain embodiments include a self-contained “cartridge” style device that is designed to work with laparoscopic robotic surgery systems, e.g., to be manipulated or actuated by the robotic surgery system to deliver devices intended to create or assist in the creation of magnetic-based compression anastomoses in the GI tract. Embodiments may include a cartridge, a series of cartridges, or other delivery method for delivery of magnetic anastomosis devices and related devices such as otomy formation/access devices (e.g., cutting or energy-based devices), otomy control devices (e.g., a grommet, or other otomy control device), implants (e.g., polymer or shape-memory material implants), etc. using surgical robots. The cutting or energy-based otomy formation features may be integral to the cartridge assembly.

Additionally, the cartridge housing may be comprised of a material capable of conducting RF energy.

### CROSS-REFERENCE TO RELATED APPLICATION(S)

This patent application is a continuation-in-part of PCT International Patent Application No. PCT/US2025/023903 entitled Systems and Methods for Delivery of Magnetic Anastomosis and Related Devices Using Surgical Robots filed Apr. 9, 2025, which claims the benefit of U.S. Provisional Ser. No. 63/632,725 entitled Systems and Methods for Delivery of Magnetic Anastomosis and Related Devices Using Surgical Robots filed Apr. 11, 2024, each of which is hereby incorporated herein by reference in its entirety.

The subject matter of this patent application may be related to the subject matter of U.S. patent application Ser. No. 18/922,011 entitled SYSTEMS, DEVICES, AND METHODS FOR DELIVERY OF OTOMY SUPPORT AND ANASTOMOSIS CREATION DEVICES filed Oct. 21, 2024 (U.S. Patent Application Publication No. US 2025/0114095—Attorney Docket No. 121326-12604, which is a continuation of International Patent Application No. PCT/US2024/047958 filed Sep. 23, 2024 (International Publication No. WO 2025/064986—Attorney Docket No. 121326-12603, which claims the benefit of United States Provisional Ser. No. 63/539,926 filed Sep. 22, 2023, each of which relates generally to a universal delivery device and is hereby incorporated herein by reference in its entirety.

The subject matter of this patent application also may be related to the subject matter of commonly-owned U.S. patent application Ser. No. 18/229,988 entitled MAGNETIC COMPRESSION ANASTOMOSIS DEVICES WITH MULTIPIECE INTERNAL VERTEBRAE SUPPORT STRUCTURES filed Aug. 3, 2023 (U.S. Patent Application Publication No. US 2024/0065694—Attorney Docket No. 121326-12101), which claims the benefit of U.S. Provisional Ser. No. 63/400,904 filed Aug. 25, 2022, each of which relates generally to magnetic compression anastomosis devices referred to herein as Flexagon™ Version 2 devices and is hereby incorporated by reference in its entirety.

The subject matter of this patent application also may be related to the subject matter of commonly-owned U.S. patent application Ser. No. 19/029,652 entitled MAGNETIC COMPRESSION ANASTOMOSIS DEVICE WITH MULTIPIECE VERTEBRA filed Jan. 17, 2025 (U.S. Patent Application Publication No. 2025/0160836—Attorney Docket No. 121326-12206), which is a continuation-in-part of U.S. patent application Ser. No. 18/230,066 entitled MAGNETIC COMPRESSION ANASTOMOSIS DEVICE WITH MULTIPIECE VERTEBRA filed Aug. 3, 2023 (U.S. Patent Application Publication No. US 2024/0041460 issued as U.S. Pat. No. 12,201,300 on Jan. 21, 2025—Attorney Docket No. 121326-12201), which claims the benefit of U.S. Provisional Patent Application No. 63/395,570 filed Aug. 5, 2022, each of which is relates generally to magnetic compression anastomosis devices referred to herein as Flexagon™ Version 1 devices and is hereby incorporated by reference in its entirety.

The subject matter of this patent application also may be related to the subject matter of commonly-owned U.S. patent application Ser. No. 19/029,809 entitled SYSTEMS AND METHODS FOR PRESERVING AND MANIPULATING OF ACUTE OTOMIES filed May 22, 2025 (U.S. Patent Application Publication No. US 2025/0160837—Attorney Docket No. 121326-12004), which is a continuation-in-part of U.S. patent application Ser. No. 18/384,022 entitled SYSTEMS AND METHODS FOR PRESERVING AND MANIPULATING OF ACUTE OTOMIES filed Oct. 26, 2023 (U.S. Patent Application Publication No. US 2024/0138839—Attorney Docket No. 121326-12003), which claims the benefit of commonly-owned U.S. Provisional Ser. No. 63/419,509 entitled SYSTEMS AND METHODS FOR PRESERVING AND MANIPULATING OF ACUTE OTOMIES filed Oct. 26, 2022 and commonly-owned United States Provisional Patent Application No. 63/435,724 entitled SYSTEMS AND METHODS FOR PRESERVING AND MANIPULATING OF ACUTE OTOMIES filed Dec. 28, 2022, each of which relates generally to otomy control devices referred to herein as OtoLoc™ otomy control devices and is hereby incorporated herein by reference in its entirety.

### FIELD OF THE INVENTION

The invention generally relates to robotic surgery, and, more particularly, to systems and methods for delivery of magnetic anastomosis devices and related devices such as otomy formation/access devices (e.g., cutting or energy-based devices), otomy control devices, implants (e.g., polymer or shape-memory material implants), etc. using surgical robots.

### BACKGROUND OF THE INVENTION

Robotic-enabled surgery or robotic-facilitated surgery is a rapidly growing field in medical device technology. Currently, robotic surgical systems such as the Intuitive Surgical Da Vinci™ robot or the Medtronic Hugo™M robot are capable of cutting, ablating/coagulating and otherwise manipulating tissue(s) either intra or extra-corporeally. These robotic systems may be used to create an anastomosis by suturing or stapling tissues together. Additionally, the devices used to accomplish the anastomosis may be specific to certain brand of surgical robot, decreasing widespread adoption of technology.

Anastomosis creation using magnets offers several advantages over traditional sutures or staples, even when the sutures or staples are applied via surgical robots. Magnetically created anastomoses, have been shown to reduce chronic inflammatory reaction and bacterial ingress and deliver a healed anastomosis that is mechanically superior compared to a staple-based anastomoses while requiring less surgical time than a suture-based anastomosis.

### SUMMARY OF VARIOUS EMBODIMENTS

In accordance with one embodiment of the invention, a cartridge for robotic delivery of otomy and/or anastomosis implants includes a chassis having a deployment channel containing at least one implant selected from the group consisting of an otomy control device and a magnetic compression anastomosis device; a deployment mechanism configured to control deployment of the at least one implant from a distal end of the deployment channel; and at least one actuation element accessible from outside the chassis and configured for operation by at least one robotic surgery system, each actuation element configured to actuate at least one deployment operation of the deployment mechanism.

In various alternative embodiments, the at least one actuation element may be configured for operation across multiple separate robotic surgery systems or may be configured for operation by a specific robotic surgery system. The chassis may be configured to be held by one set of robotically controlled graspers while the at least one actuation element is being operated by another set of robotically controlled graspers and/or may be configured to fit onto one set of robotically controlled graspers while the at least one actuation element is being operated by another set of robotically controlled graspers. The at least one actuation element may include radially extended tabs or other features that facilitate manipulation and actuation by robotic graspers, coaxial or noncoaxial extended pushrods or other features that facilitate manipulation and actuation by robotic graspers, a rotary feature that drives a shuttle (e.g., leadscrew) or other features that facilitate manipulation and actuation by robotic graspers, and/or a trigger mechanism. The chassis and actuation element may be configured for use with both a universal delivery device and at least one robotic surgery system.

In various embodiments, the at least one implant may include an otomy control device (e.g., a grommet otomy control device) and/or a magnetic compression anastomosis device (e.g., a self-assembling magnetic compression anastomosis device or a single piece, bar-style magnet. The at least one implant may include only one implant or may include a distal implant and a proximal implant, wherein the deployment mechanism is configured to deploy the distal implant followed by the proximal implant (e.g., to perform a first deployment operation that deploys the distal implant and to perform a second deployment operation that deploys the proximal implant, wherein the second deployment operation requires a separate actuation from the first deployment operation). In some embodiments, the distal implant may be an otomy control device and the proximal implant may be a magnetic compression anastomosis device, whereas in other embodiments, the distal implant may be a magnetic compression anastomosis device and the proximal implant may be an otomy control device. The deployment mechanism may include a spring-loaded mechanism, a pusher configured to push the at least one implant from the distal end of the deployment channel, a ratcheting feature to control advancement of the at least one implant, a linkage drive mechanism for controlling actuation and release operations, and/or a cable drive mechanism for controlling actuation and release operations. The distal tip of the chassis may be configured to facilitate access into and/or dilation of an otomy. The cartridge may be configured for delivery through a trocar. The cartridge may include at least one separable section (e.g., a peel-away or tear-away section) for controlled deployment of at least a portion of an implant.

Various alternative embodiments include a system including at least one robotic surgery system and a cartridge, wherein the cartridge is attached to the at least one robotic surgery system for at least controlling the at least one actuation element to deliver the at least one implant from the cartridge.

Various alternative embodiments also include a robotic surgical method comprising providing a cartridge; attaching the cartridge to the at least one robotic surgery system; and operating the surgical robot to control the at least one actuation element to deliver the at least one implant from the cartridge.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A-1B show a cartridge configured to fit onto a first robotic laparoscopic grasper, with an actuation point configured to be held/actuated by a second robotic grasper to deliver a self-forming magnet, shown with the self-forming magnet partially deployed (FIG. 1A) and almost fully deployed (FIG. 1B).

FIGS. 2A-2B show a cartridge configured to fit onto a first robotic laparoscopic grasper, with an actuation point configured to be held/actuated by a second robotic grasper and deliver an otomy protection device, shown with the otomy protection device completely constrained (FIG. 2A) and partially deployed (FIG. 2B).

FIGS. 3A-3B show a cartridge configured to be held by a first robotic laparoscopic grasper, with an actuation point configured to be held/actuated by a second robotic grasper to deliver a self-forming magnet, shown with the self-forming magnet fully constrained (FIG. 3A) and partially deployed (FIG. 3B).

FIGS. 4A-4B show one method for attaching a cartridge onto a robotic grasper (FIG. 4A) and locking the cartridge in place (FIG. 4B).

FIGS. 5A-5C show a cartridge configured to be held by a first robotic laparoscopic grasper, with an actuation point configured to be held/actuated by a second robotic grasper and deliver both an otomy protection device followed by a self-forming magnet, shown with the otomy protection device and self-forming magnet fully constrained (FIG. 5A), a partially deployed otomy protection device (FIG. 5B), and a partially deployed self-forming magnet (FIG. 5C).

FIG. 6 shows a cartridge including a chassis that is used in a peel-away or tear-away fashion.

FIG. 7 shows a multi-piece cartridge chassis allowing for multi staged deployment.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Certain embodiments of the invention include a self-contained “cartridge” style device that is designed to work with laparoscopic robotic surgery systems, e.g., to be manipulated or actuated by the robotic surgery system to deliver devices intended to create or assist in the creation of magnetic-based compression anastomoses in the GI tract. Embodiments may include a cartridge, a series of cartridges, or other delivery method for delivery of magnetic anastomosis devices (e.g., self-assembling magnetic compression anastomosis devices or bar-style magnets including bar-style magnets with a through-hole or lumen or magnetic compression anastomosis devices containing bioabsorbable or bio-fragmentable materials) and related devices such as otomy formation/access devices (e.g., cutting or energy based devices), otomy control devices (e.g., a grommet, or other otomy control device), implants (e.g., polymer or shape-memory material implants), etc. using surgical robots. The cutting or energy-based otomy formation features (e.g., RF or heat based energy devices) may be integral to the cartridge assembly, e.g., as opposed to being part of the robotic system, the implants being delivered, or an ancillary device. Additionally, the cartridge housing may be comprised of a material capable of conducting RF energy.

A cartridge may consist of a chassis with an attachment point designed for a surgical robot end effector (often referred to herein as a “grasper” although embodiments can be configured to operate with any types of robot attachments), a self-contained implant or implants, and deployment features for an implant or implants. Cartridges may be flexible, articulating, or rigid.

The implant(s) are generally radially and or axially constrained with the cartridge or within components or assemblies within the cartridge, e.g., within a cannula or tube within the cartridge body. The various elements within the cartridge are allowed to slide adjacent to or telescope within one another to push or pull on the implant(s) to advance them out of the cartridge. Some embodiments may include features to retract the implants back into the cartridge. The cartridge is configured to be held by the robotic graspers either at a point or points along the cartridge body or by features on the actuating elements of the cartridge.

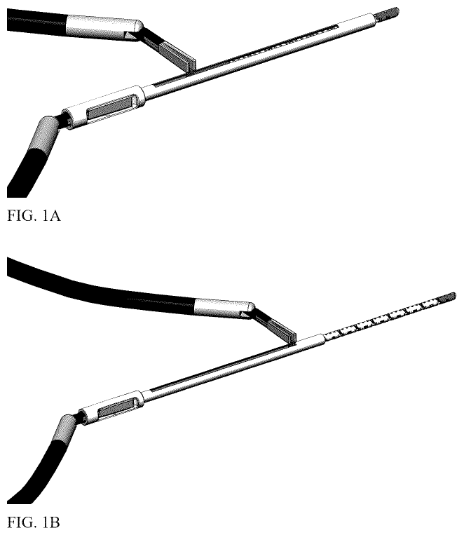

Example 1: A cartridge configured to fit onto a first robotic laparoscopic grasper, with an actuation point configured to be held/actuated by a second robotic grasper to deliver a magnetic compression anastomosis device such as a self-forming magnet (e.g., pursuant to Potential Claim P2 below), e.g., by pushing the magnet out of the cartridge. One exemplary embodiment with a self-forming magnet partially deployed is shown in FIG. 1A and with the self-forming magnet almost fully deployed is shown in FIG. 1B. It should be noted that the cartridge alternatively could include a delivery system that can be actuated by the second robotic grasper, e.g., a spring-loaded mechanism that can be actuated such as by actuating a trigger mechanism to push the self-forming magnet out of the cartridge.

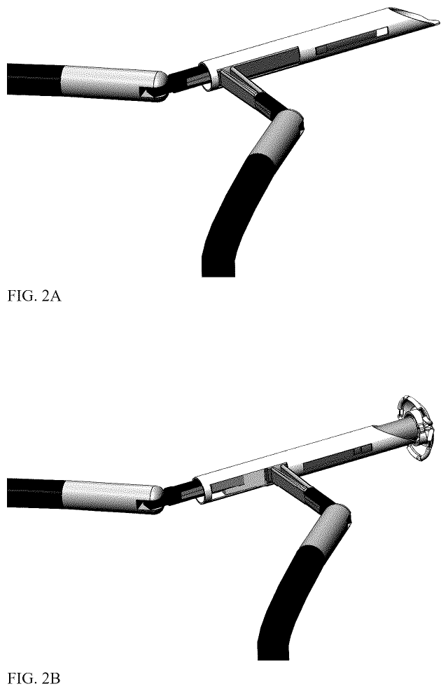

Example 2: A cartridge configured to fit onto a first robotic laparoscopic grasper, with an actuation point configured to be held/actuated by a second robotic grasper and deliver an otomy protection device (e.g., pursuant to Potential Claim P3 below), e.g., by pushing the otomy protection device out of the cartridge. One exemplary embodiment with the otomy protection device completely constrained is shown in FIG. 2A and with the otomy protection device partially deployed is shown in FIG. 2B. Note the beveled/formed tip in this embodiment for access and dilation of an otomy. It should be noted that the cartridge alternatively could include a delivery system that can be actuated by the second robotic grasper, e.g., a spring-loaded mechanism that can be actuated such as by actuating a trigger mechanism to push the otomy protection device out of the cartridge.

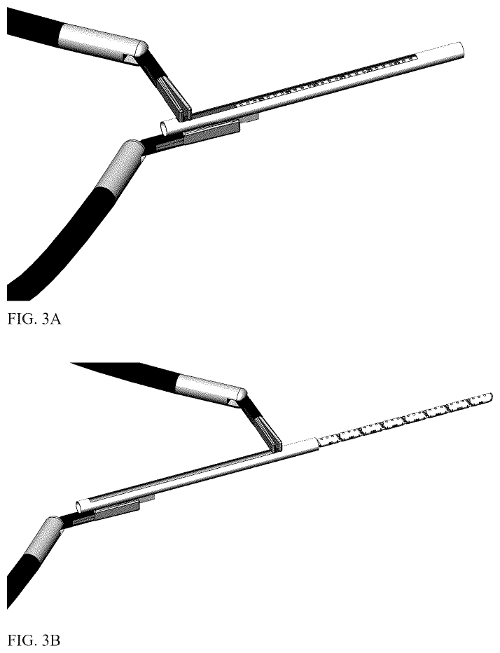

Example 3: A cartridge configured to be held by a first robotic laparoscopic grasper, with an actuation point configured to be held/actuated by a second robotic grasper to deliver a magnet such as a self-forming magnet, e.g., by pushing the magnet out of the cartridge. One exemplary embodiment with the self-forming magnet fully constrained is shown in FIG. 3A and with the self-forming magnet partially deployed is shown in FIG. 3B. It should be noted that the cartridge alternatively could include a delivery system that can be actuated by the second robotic grasper, e.g., a spring-loaded mechanism that can be actuated such as by actuating a trigger mechanism to push the self-forming magnet out of the cartridge.

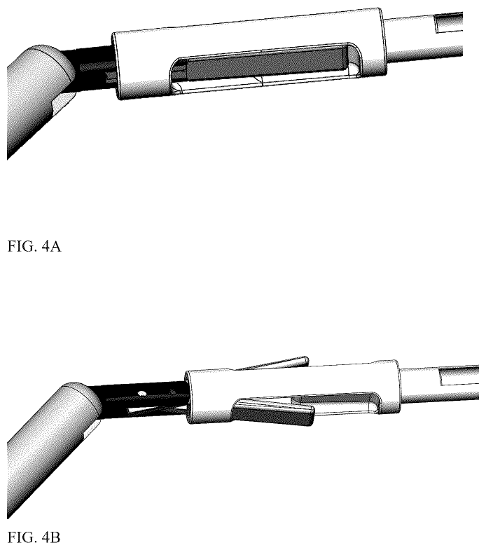

Example 4: An apparatus and method for attaching a cartridge onto a robotic grasper (e.g., as depicted in FIG. 4A) and locking the cartridge in place (e.g., as depicted in FIG. 4B).

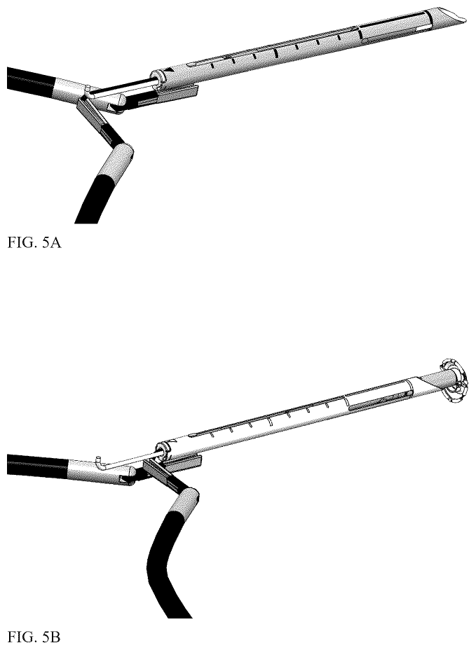

Example 5: A cartridge configured to be held by a first robotic laparoscopic grasper, with an actuation point configured to be held/actuated by a second robotic grasper and deliver both an otomy protection device followed by a magnet such as a self-forming magnet (e.g., pursuant to Potential Claim P1 below). One exemplary embodiment with the otomy protection device and self-forming magnet fully constrained is shown in FIG. 5A, with a partially deployed otomy protection device is shown in FIG. 5B, and with a partially deployed self-forming magnet is shown in FIG. 5C. Note that this exemplary embodiments includes noncoaxial pushrods for sequential actuation and deployment of the individual implants. It should be noted that, rather than pushrods, the cartridge alternatively could include a delivery system that can be actuated by the second robotic grasper, e.g., a spring-loaded mechanism that can be actuated such as by actuating a trigger mechanism to push the devices out of the cartridge in the described order.

It should be noted that the chassis of cartridges of the types described herein can include one or more separable sections to allow for controlled deployment of at least one portion of an implant.

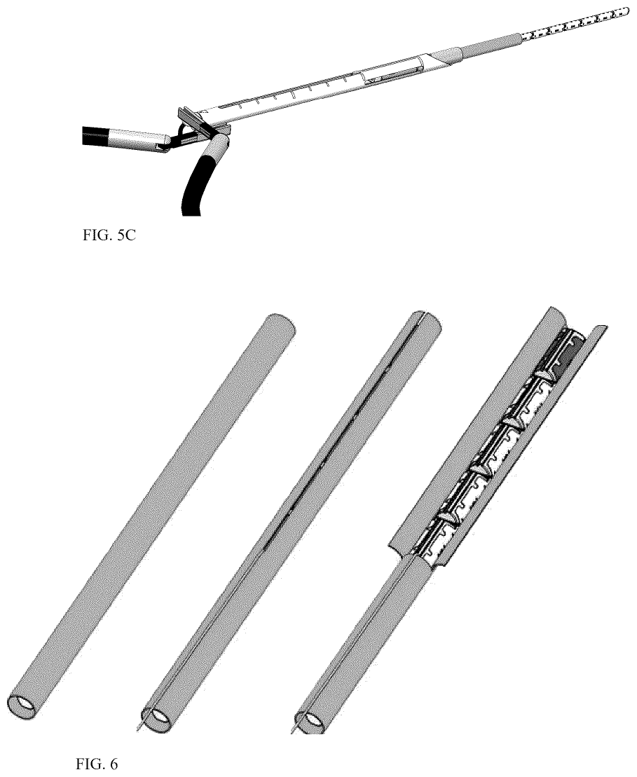

FIG. 6 shows a cartridge including a chassis having a separable section that is used in a peel-away or tear-away fashion, e.g., an implant such as an otomy control device or magnetic compression anastomosis device contained within a piece of tubing that has a rip cord or other separation mechanism. This design works by constraining the implant within a chassis that has a built-in weak point. The implant-containing chassis is delivered through a lumen, e.g., using robotic graspers. Once positioned, the weak point is broken, e.g., in a rip-cord fashion, allowing the chassis to split open and deploy some or all of the implant in a controlled manner (e.g., an entire self-assembling magnet could be deployed in this fashion, one flange of an otomy protection device could be deployed in this fashion, etc.).

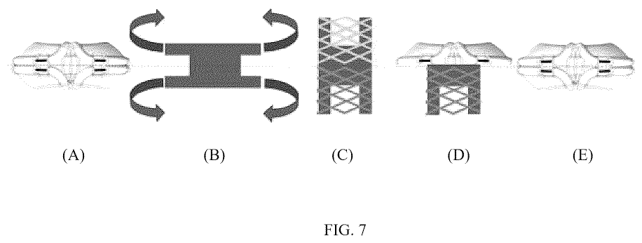

As depicted schematically in FIG. 7, certain embodiments can include multiple separable sections to allow for multi-staged deployment of one or more constrained devices. For convenience, such a device may be referred to herein as a multi-piece cartridge chassis. The design shown in FIG. 7 consists of two separable sections including a top chassis element and a bottom chassis element to deploy an otomy protection device of the type shown in FIG. 7(A), where the top and bottom chassis elements can be removed or otherwise separated in stages to deploy different parts of the implant as depicted schematically in FIG. 7(B). FIG. 7(B) depicts the top and bottom chassis elements being removed in a peel-away or tear-away fashion such as described above with reference to FIG. 6, although alternative embodiments could remove the chassis elements in other ways (e.g., sliding off, separating into two or more segments, collapsing, unwrapping, dissolving, etc.). Among other things, a multi-piece cartridge chassis allows the implant to be delivered in a constrained state, with selective chassis removal enabling precise deployment. For example, an Otoloc cartridge design could use two flexible meshes to respectively constrain the two flanges of the otomy protection device implant, as depicted schematically in FIG. 7(C). The constrained implant can be inserted into the abdominal cavity and then into a lumen. The top mesh chassis can be removed first to deploy a first flange intraluminally with the second flange still constrained within the cartridge by the bottom mesh chassis as depicted schematically in FIG. 7(D). The remaining constrained bottom mesh chassis then can be dragged out of the lumen into the abdominal cavity, where it is removed to deploy the extraluminal flange to deploy the entire otomy protection device as depicted in FIG. 7(E). Other embodiments operating under similar principles can include two or more pieces, e.g., allowing for staged deployment of different sections of an otomy protection device, different magnet segments, etc.

It should be noted that cartridges may be configured to work with both a universal delivery device (e.g., a universal delivery device of the types described in the patent applications incorporated by reference above) and the surgical robot, e.g., by including common actuation element(s) that work with both the universal delivery device and the surgical robot, or by including first actuation element(s) that work with the universal delivery device and second actuation element(s) that work with the surgical robot. The deployment mechanism of the cartridge can be configured to emulate the operation of any operation(s) performed by the universal delivery device (e.g., the actuation trigger and the release trigger operations of the universal delivery device) as discussed herein.

It should be noted that magnetic compression anastomosis devices can include self-forming devices such as a Flexagon™ device from GI Windows of the types described in the patent applications incorporated by reference above and an otomy control device can include an OTOLoc™ device from GI Windows of the types described in the patent applications incorporated by reference above.

It should be noted that cartridges of the types described herein can include one or more windows, through-holes, or other visibility features that allow the physician to view/monitor the deployment status of the at least one implant, for example, a window as shown in FIG. 1A. It also should be noted that cartridges of the types described herein can include one or more markings to indicate insertion depth.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:

P1. A cartridge-based device designed to interact with and be manipulated by a surgical robot that is capable of introducing a magnetic compression anastomosis device and an otomy protection device in tandem into a target patient anatomy.
a. The device in Claim P1 wherein the magnetic compression anastomosis device is delivered ahead of the otomy protection device.
b. The device in Claim P1 wherein the otomy protection device is delivered ahead of the magnetic compression anastomosis device.
c. The device in Claim P1 wherein the system is configured to work across multiple separate robotic surgery systems.
d. The device in Claim P1 wherein multiple versions of the system are configured to work with specific robotic surgery systems.
e. The device in Claim P1 wherein the cartridge is delivered through a trocar.
f. The device in Claim P1 wherein the distal tip of the cartridge is configured to facilitate access into and dilation of an enterotomy, a gastrotomy, a colotomy, an esophagotomy, or other “-otomy,” e.g., wherein the cartridge or cartridge components include beveled, formed, or cutting features to create the otomy and/or features for RF or other energy conduction to create the otomy.
g. The device in claim P1 wherein the magnetic compression anastomosis device is configured for a low-profile delivery and during or after delivery self-forms into a ring or other geometry creating an open lumen.
h. The device in Claim P1 wherein the cartridge assembly or some or all of its components are made of non-ferrous materials.
i. The device in Claim P1 wherein the cartridge is configured to allow retraction of a partially deployed implant.
j. The device in Claim P1 wherein the magnetic compression anastomosis device is a single piece, bar-style magnet.
k. The device in Claim P1 wherein the magnetic compression anastomosis device is a single piece, bar-style magnet with a through-hole or lumen.
l. The device in Claim P1 wherein the cartridge is configured to be held by one set of robotically controlled graspers while being actuated by another set of robotically controlled graspers.
m. The device in Claim P1 wherein the cartridge is configured to fit onto one set of robotically controlled graspers while being actuated by another set of robotically controlled graspers.
n. The device in Claim P1 wherein the cartridge is sufficiently short that it is fully intracorporeal during manipulation of the cartridge and delivery of the implants.
o. The device in Claim P1 wherein the cartridge is sufficiently long that it extends extracorporeally and can be manipulated by robotically controlled graspers that are outside the body.
p. The device in Claim P1 wherein the actuation interface consists of radially extended tabs or other features that facilitate manipulation and actuation by robotic graspers.
q. The device in Claim P1 wherein the actuation interface consists of coaxial or noncoaxial extended pushrods or other features that facilitate manipulation and actuation by robotic graspers.
r. The device in Claim P1 wherein the actuation interface consists of a rotary feature that drives a shuttle (e.g. leadscrew) or other features that facilitate manipulation and actuation by robotic graspers.
s. The device in Claim P1 wherein the overall cartridge functions as a rigid member.
t. The device in Claim P1 wherein the cartridge is flexible, or contains a mechanism to allow one on more direction of articulation or rotation.
u. The device in Claim P1 wherein a means of manipulating the magnetic implant after it is deployed into the target anatomy (e.g. a tether or control suture) is contained within the cartridge in such a way as to facilitate a controlled release of either or both the magnetic implant and the controlling element.
v. The device in Claim P1 wherein the magnetic compression anastomosis device incorporates a bioabsorbable or bio-fragmentable material.

P2. A device designed to interact with and be manipulated by a surgical robot that is capable of introducing only a magnetic compression anastomosis device into a target patient anatomy.
a. The device in Claim P2 wherein the system is configured to work across multiple separate robotic surgery systems.
b. The device in Claim P2 wherein multiple versions of the system are configured to work with specific robotic surgery systems.
c. The device in Claim P2 wherein the cartridge is delivered through a trocar.
d. The device in Claim P2 wherein the distal tip of the cartridge is configured to facilitate access into and dilation of an enterotomy, a gastrotomy, a colotomy, an esophagotomy, or other “-otomy,” e.g., wherein the cartridge or cartridge components include beveled, formed, or cutting features to create the otomy and/or features for RF or other energy conduction to create the otomy.
e. The device in claim P2 wherein the magnetic compression anastomosis device is configured for a low-profile delivery and during or after delivery self-forms into a ring or other geometry creating an open lumen.
f. The device in Claim P2 wherein the magnetic compression anastomosis device is a single piece, bar-style magnet.
g. The device in Claim P2 wherein the magnetic compression anastomosis device is a single piece, bar-style magnet with a through-hole or lumen.
h. The device in Claim P2 wherein the cartridge assembly or some or all of its components are made of non-ferrous materials.
i. The device in Claim P2 wherein the cartridge is configured to allow retraction of a partially deployed implant.
j. The device in Claim P2 wherein the cartridge is configured to be held by one set of robotically controlled graspers while being actuated by another set of robotically controlled graspers.
k. The device in Claim P2 wherein the cartridge is configured to fit onto one set of robotically controlled graspers while being actuated by another set of robotically controlled graspers.
l. The device in Claim P2 wherein the cartridge is sufficiently short that it is fully intracorporeal during manipulation of the cartridge and delivery of the implants.
m. The device in Claim P2 wherein the cartridge is sufficiently long that it extends extracorporeally and can be manipulated by robotically controlled graspers that are outside the body.
n. The device in Claim P2 wherein the actuation interface consists of radially extended tabs or other features that facilitate manipulation and actuation by robotic graspers.
o. The device in Claim P2 wherein the actuation interface consists of coaxial or noncoaxial extended pushrods or other features that facilitate manipulation and actuation by robotic graspers.
p. The device in Claim P2 wherein the actuation interface consists of a rotary feature that drives a shuttle (e.g. leadscrew) or other features that facilitate manipulation and actuation by robotic graspers.
q. The device in Claim P2 wherein the overall cartridge functions as a rigid member.
r. The device in Claim P2 wherein the cartridge is flexible, or contains a mechanism to allow one on more direction of articulation or rotation.
s. The device in Claim P2 wherein a means of manipulating the magnetic implant after it is deployed into the target anatomy (e.g. a tether or control suture) is contained within the cartridge in such a way as to facilitate a controlled release of either or both the magnetic implant and the controlling element.
t. The device in Claim P2 wherein the magnetic compression anastomosis device incorporates a bioabsorbable or bio-fragmentable material.

P3. A device designed to interact with and be manipulated by a surgical robot that is capable of introducing only an otomy protection device into a target patient anatomy.
a. The device in Claim P3 wherein the system is configured to work across multiple separate robotic surgery systems.
b. The device in Claim P3 wherein multiple versions of the system are configured to work with specific robotic surgery systems.
c. The device in Claim P3 wherein the cartridge is delivered through a trocar.
d. The device in Claim P3 wherein the distal tip of the cartridge is configured to facilitate access into and dilation of an enterotomy, a gastrotomy, a colotomy, an esophagotomy, or other “-otomy,” e.g., wherein the cartridge or cartridge components include beveled, formed, or cutting features to create the otomy and/or features for RF or other energy conduction to create the otomy.
e. The device in Claim P3 wherein the cartridge is configured to be held by one set of robotically controlled graspers while being actuated by another set of robotically controlled graspers.
f. The device in Claim P3 wherein the cartridge is configured to fit onto one set of robotically controlled graspers while being actuated by another set of robotically controlled graspers.
g. The device in Claim P3 wherein the cartridge is sufficiently short that it is fully intracorporeal during manipulation of the cartridge and delivery of the implants.
h. The device in Claim P3 wherein the cartridge is configured to allow retraction of a partially deployed implant.
i. The device in Claim P3 wherein the cartridge is sufficiently long that it extends extracorporeally and can be manipulated by robotically controlled graspers that are outside the body.
j. The device in Claim P3 wherein the actuation interface consists of radially extended tabs or other features that facilitate manipulation and actuation by robotic graspers.
k. The device in Claim P3 wherein the actuation interface consists of coaxial or noncoaxial extended pushrods or other features that facilitate manipulation and actuation by robotic graspers.
l. The device in Claim P3 wherein the actuation interface consists of a rotary feature that drives a shuttle (e.g. leadscrew) or other features that facilitate manipulation and actuation by robotic graspers.
m. The device in Claim P3 wherein the overall cartridge functions as a rigid member.
n. The device in Claim P3 wherein the cartridge is flexible, or contains a mechanism to allow one on more direction of articulation or rotation.
o. The device in Claim P3 wherein the magnetic compression anastomosis device incorporates a bioabsorbable or bio-fragmentable material.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. A cartridge for robotic delivery of otomy and/or anastomosis implants, the cartridge comprising:
a chassis having a deployment channel containing at least one implant selected from the group consisting of an otomy control device and a magnetic compression anastomosis device;
a deployment mechanism configured to control deployment of the at least one implant from a distal end of the deployment channel; and
at least one actuation element accessible from outside the chassis and configured for operation by at least one robotic surgery system, each actuation element configured to actuate at least one deployment operation of the deployment mechanism.

2. The cartridge of claim 1, wherein the at least one actuation element is configured for operation across multiple separate robotic surgery systems.

3. The cartridge of claim 1, wherein the at least one actuation element is configured for operation by a specific robotic surgery system.

4. The cartridge of claim 1, wherein the chassis is configured to be held by or fit onto one set of robotically controlled graspers while the at least one actuation element is being operated by another set of robotically controlled graspers, optionally wherein the chassis and actuation element are configured for use with both a universal delivery device and at least one robotic surgery system.

5. (canceled)

6. The cartridge of claim 1, wherein the at least one actuation element comprises at least one of:
radially extended tabs or other features that facilitate manipulation and actuation by robotic graspers;
coaxial or noncoaxial extended pushrods or other features that facilitate manipulation and actuation by robotic graspers;
a rotary feature that drives a shuttle (e.g., leadscrew) or other features that facilitate manipulation and actuation by robotic graspers; or
a trigger mechanism.

7. (canceled)

8. (canceled)

9. (canceled)

10. (canceled)

11. The cartridge of claim 1, wherein the at least one implant comprises an otomy control device, optionally wherein the otomy control device is a grommet otomy control device.

12. (canceled)

13. The cartridge of claim 1, wherein the at least one implant comprises a magnetic compression anastomosis device, optionally wherein at least one of:
the magnetic compression anastomosis device is a self-assembling magnetic compression anastomosis device, optionally wherein the magnetic compression anastomosis device is configured for a low-profile delivery (e.g., a substantially linear configuration within the deployment channel) and during or after delivery self-forms into a ring or other geometry to create an open lumen;
the magnetic compression anastomosis device is a single piece, bar-style magnet, optionally wherein the single piece, bar-style magnet comprises a through-hole or lumen; or
the magnetic compression anastomosis device incorporates a bioabsorbable or bio-fragmentable material.

14. (canceled)

15. (canceled)

16. (canceled)

17. The cartridge of claim 1, wherein the at least one implant includes only one implant.

18. The cartridge of claim 1, wherein the at least one implant includes a distal implant and a proximal implant, wherein the deployment mechanism is configured to deploy the distal implant followed by the proximal implant, optionally wherein the deployment mechanism is configured to perform a first deployment operation that deploys the distal implant and to perform a second deployment operation that deploys the proximal implant, wherein the second deployment operation requires a separate actuation from the first deployment operation.

19. (canceled)

20. The cartridge of claim 18, wherein the distal implant is an otomy control device and the proximal implant is a magnetic compression anastomosis device, optionally wherein at least one of:
the cartridge contains the otomy control device distal to the magnetic compression anastomosis device without otomy alignment, wherein the cartridge deploys the otomy control device and then requires access of the otomy control device prior to deployment of the magnetic compression anastomosis device through the otomy control device;
the cartridge contains the otomy control device distal to the magnetic compression anastomosis device with thru deployment, wherein the cartridge deploys a distal flange of the otomy control device and then deploys the magnetic compression anastomosis device through the otomy control device and then deploys a proximal flange of the otomy control device; or
the cartridge contains the otomy control device distal to the magnetic compression anastomosis device with alignment, wherein the cartridge deploys the otomy control device and maintains a connection with the otomy control device for deployment of the magnetic compression anastomosis device through the otomy control device and subsequent release of the otomy control device.

21. (canceled)

22. (canceled)

23. (canceled)

24. The cartridge of claim 18, wherein the distal implant is a magnetic compression anastomosis device and the proximal implant is an otomy control device.

25. The cartridge of claim 1, wherein the deployment mechanism comprises at least one of:
a spring-loaded mechanism;
a pusher configured to push the at least one implant from the distal end of the deployment channel;
a ratcheting feature to control advancement of the at least one implant;
a linkage drive mechanism for controlling actuation and release operations; or
a cable drive mechanism for controlling actuation and release operations.

26. (canceled)

27. (canceled)

28. (canceled)

29. (canceled)

30. The cartridge of claim 1, wherein at least one of:
a distal tip of the chassis is configured to facilitate access into and/or dilation of an enterotomy, a gastrotomy, a colotomy, an esophagotomy, or other otomy, optionally wherein the cartridge or cartridge components include beveled, formed, or cutting features to create the otomy and/or features for RF or other energy conduction to create the otomy;
at least the chassis is made of non-ferrous material;
the chassis is rigid;
the chassis is flexible;
the chassis includes a mechanism that allows one or more direction of articulation or rotation; or
the cartridge is configured for delivery through a trocar.

31. (canceled)

32. The cartridge of claim 1, wherein at least one of the deployment mechanism or the at least one actuation element is further configured to allow retraction of a partially deployed implant.

33. The cartridge of claim 1, further comprising at least one element for manipulating the at least one implant before and/or after the implant is deployed, optionally wherein the at least one element comprises a tether or control suture.

34. (canceled)

35. The cartridge of claim 1, wherein the cartridge is configured to be fully intracorporeal during manipulation of the cartridge and delivery of the at least one implant.

36. The cartridge of claim 1, wherein the cartridge is configured to extend extracorporeally and can be manipulated by robotically controlled graspers that are outside the body.

37. (canceled)

38. (canceled)

39. (canceled)

40. The cartridge of claim 1, wherein the at least one implant includes three or more implants.

41. The cartridge of claim 1 wherein the chassis includes windows, through-holes, one or more markings, or other features that allow the physician to view/monitor the deployment status of the at least one implant and/or insertion depth.

42. (canceled)

43. (canceled)

44. The cartridge of claim 1, wherein the chassis includes at least one separable section for controlled deployment of at least a portion of an implant, optionally wherein the chassis comprises a plurality of separable sections that can be actuated in sequential steps for deployment.

45. The cartridge of claim 44, wherein the at least one separable section is a peel-away or tear-away section, optionally wherein the peel-away or tear-away section is configured to split a portion of the chassis in a controlled manner to enable staged deployment of at least portion of an implant.

46. (canceled)

47. (canceled)

48. A system comprising:
at least one robotic surgery system; and
a cartridge comprising:
a chassis having a deployment channel containing at least one implant selected from the group consisting of an otomy control device and a magnetic compression anastomosis device;
a deployment mechanism configured to control deployment of the at least one implant from a distal end of the deployment channel; and
at least one actuation element accessible from outside the chassis and configured for operation by at least one robotic surgery system, each actuation element configured to actuate at least one deployment operation of the deployment mechanism, wherein the cartridge is attached to the at least one robotic surgery system for at least controlling the at least one actuation element to deliver the at least one implant from the cartridge.

49. A robotic surgical method comprising:
providing a cartridge comprising:
a chassis having a deployment channel containing at least one implant selected from the group consisting of an otomy control device and a magnetic compression anastomosis device;
a deployment mechanism configured to control deployment of the at least one implant from a distal end of the deployment channel; and
at least one actuation element accessible from outside the chassis and configured for operation by at least one robotic surgery system, each actuation element configured to actuate at least one deployment operation of the deployment mechanism;
attaching the cartridge to the at least one robotic surgery system; and
operating the surgical robot to control the at least one actuation element to deliver the at least one implant from the cartridge.
